(19) United States
(12) Patent Application Publication
(10) Pub. No.: US 2012/0130483 A1
(43) Pub. Date: May 24, 2012

(54) TUBULAR IMPLANT FOR REPLACING NATURAL BLOOD VESSELS

(75) Inventors: Helmut Goldmann, Kassel (DE); Christof Merckle, Mannheim (DE); Dietmar Probst, Melsungen (DE); Dennis Langanke, Tubingen (DE)

(73) Assignee: AESCULAP AG, Tuttlingen/Donau (DE)

(21) Appl. No.: 13/387,410

(22) PCT Filed: Mar. 18, 2010
(86) PCT No.: PCT/EP2010/001721

§ 371 Date: Jan. 27, 2012

(30) Foreign Application Priority Data
Jul. 31, 2009 (DE) 10 2009 037 134.6

(51) Int. Cl. A61F 2/82; B05D 1/18; B05D 1/02; B05D 7/00

(52) U.S. Cl. 623/1.46; 427/2.25

(57) ABSTRACT

A tubular implant that replaces natural blood vessels includes a prefabricated vascular prosthesis with an internal surface, an external surface and a wall, wherein the internal and/or external surface of the prefabricated vascular prosthesis has a polyurethane coating.

### RELATED APPLICATIONS

This is a §371 of International Application No. PCT/EP2010/001721, with an international filing date of Mar. 18, 2010 (WO 2011/012178 A2, published Feb. 3, 2011), which is based on German Patent Application No. 10 2009 037 134.6, filed Jul. 31, 2009, the subject matter of which is incorporated by reference.

### TECHNICAL FIELD

This disclosure relates to a tubular implant for replacing natural blood vessels and a method of production thereof.

### BACKGROUND

The replacement of sections of hollow organs, in particular of blood vessels, in humans and animals using artificial vascular prostheses is the subject of vascular surgery. The vascular prostheses used can consist of both textile and nontextile material.

As a rule, vascular prostheses are of porous design to permit ingrowth of body cells and body tissue for secondary anchoring of the prostheses and to permit the attainment of conditions that are as natural as possible. However, as these pores can lead, after implantation of the prostheses, to undesirably high losses of body fluids, especially blood, as a general rule the pores are sealed with a resorbable material, which is replaced successively with ingrowing tissue. Crosslinked gelatin (EP 0 237 037 B1) or crosslinked collagen (DE 14 91 218 A2, U.S. Pat. No. 4,167,045, DE 35 03 127 and DE 35 03 126 A1) are mainly used for sealing vascular prostheses. The problem with these sealing agents is that they are of xenogenous, as a rule bovine, origin. As a result, despite all precautions, animal disease pathogens can get into the patient's body, with the risk of postoperative complications. Even surgical reinterventions may be necessary.

Vascular prostheses that do not have any xenogenous sealing materials often have the disadvantage that they are expensive to produce (Ann Thorac. Surg. 2008, 85, 305 to 309).

Another problem connected with conventional prostheses is the occurrence of so-called “stitch track” hemorrhages during suturing-in of the vascular prostheses. Such stitch track hemorrhages can be attributed to dilation of the prosthesis wall by the needle used to suture the vascular prosthesis and partially also to detachment of sealing materials from the external prosthesis surface. This can lead to an undesirable blood loss, which is critical for the patient affected.

Therefore, it could be helpful to provide an implant that overcomes the known shortcomings, in particular permitting implantation that is as impervious to blood as possible and that reduces the risk of surgical reinterventions. Moreover, it could be helpful to provide an implant that is as simple as possible in its production and handling.

### SUMMARY

We provide a tubular implant that replaces natural blood vessels including a prefabricated vascular prosthesis with an internal surface, an external surface and a wall, wherein the internal and/or external surface of the prefabricated vascular prosthesis has a polyurethane coating.

We also provide a method of producing the tubular implant that replaces natural blood vessels, wherein the prefabricated vascular prosthesis with the internal and external surfaces and the wall is coated with polyurethane on the internal and/or external surface.

### BRIEF DESCRIPTION OF THE DRAWINGS

FIG. 1 shows a SEM photograph of an implant based on a knitted vascular prosthesis, with only its external surface coated with polyurethane (FIG. 1a). In contrast, the internal surface of the knitted vascular prosthesis is essentially free from a polyurethane coating (FIG. 1b). FIG. 1c shows the longitudinal section of the implant.

FIG. 2 shows a SEM photograph of an implant based on a woven vascular prosthesis, with only its external surface coated with polyurethane (FIG. 2a). In contrast, the internal surface of the woven vascular prosthesis is essentially free from a polyurethane coating (FIG. 2b). FIG. 2c shows the longitudinal section of the implant.

FIG. 3 shows a SEM photograph of an implant based on an expanded polytetrafluoroethylene (ePTFE) prosthesis with only its internal surface coated with polyurethane. In contrast, the external surface of the prosthesis is essentially free from a polyurethane coating. FIG. 3 shows that the polyurethane coating partially penetrates into the node and fibril structure of the ePTFE prosthesis yielding a firm composite structure.

### DETAILED DESCRIPTION

Our implant is a tubular or hose-shaped implant for replacing natural blood vessels comprising a prefabricated vascular prosthesis (basic prosthesis) with an internal surface and an external surface and a wall, wherein the internal surface and/or external surface of the prefabricated vascular prosthesis is coated with polyurethane.

A prefabricated vascular prosthesis is to be understood as a prosthesis, which can already be used per se, as a rule as a so-called “interposition graft” to replace natural blood vessels, in particular arterial blood vessels, or as a so-called “bypass” to circumvent blocked sections of natural blood vessels, in particular arterial blood vessels. The prefabricated vascular prosthesis can therefore in particular be a vascular prosthesis.

In other words, we provide a polyurethane-coated vascular prosthesis, wherein the polyurethane coating is formed on the internal and/or external surface of prefabricated vascular prosthesis.

We surprisingly found that prefabricated vascular prostheses coated with polyurethane can be implanted so that they are essentially impervious to blood, i.e., without undesirable blood losses and seroma formations after implantation. In other words, polyurethane is particularly suitable as a sealing agent for vascular prostheses. The use of xenogenous material is no longer necessary, meaning that the associated risks can be avoided. As a rule, the coating is therefore formed over the whole area of and in particular provides sealing on the internal and/or external surface of the prefabricated prosthesis. Polyurethane itself is a biocompatible material that is widely accepted in the medical field. As certain polyurethanes are in addition nonresorbable materials, the implant only triggers mild tissue reactions, with the result that the incorporation of the implant is accelerated.

Preferably, polyurethane forms the main constituent of the coating. It can be envisaged that in addition to polyurethane, the coating can also contain other constituents discussed in more detail below. Preferably the coating has a proportion of polyurethane of at least 80 wt. %, in particular at least 90 wt. %, preferably at least 95 wt. %, especially preferably at least 98 wt. %, based on the total weight of the coating. In particular, the coating consists essentially only of polyurethane. This means that the proportion of polyurethane in the coating can be at least 99 wt. %, based on the total weight of the coating.

The polyurethane coating provided may be porous, preferably with open pores. An open-pored coating has the advantage that connective tissue cells, so-called “fibroblasts,” can grow into the implant from outside and can secrete substances that are responsible for the structure of connective tissue, in particular collagen, reticulin, fibronectin and/or elastin. As a result, on the one hand, the implant is firmly anchored in the patient's body. On the other hand, this means that the implant can reproduce or mimic, in an especially advantageous manner, the original anatomical conditions in the implantation region.

Pores of the coating may have a resorbable material, in particular a resorbable polymer. In particular, pores of the coating are filled at least partially, preferably completely, with a resorbable material, in particular a resorbable polymer. Preferred resorbable materials are polyhydroxyalkanoates. For example, the resorbable material can be selected from the group comprising polyglycolide, polylactide, polytrimethylene carbonate, poly-para-dioxanone, poly-ε-caprolactone, poly-3-hydroxybutyrate, poly-4-hydroxybutyrate, copolymers thereof and combinations, in particular blends, thereof.

A copolymer is a polymer that is composed of at least two different monomer units.

A polyurethane may encompass a polyurethane homo- and/or copolymer.

Depending on the resorption rate or resorption time of the material, there is ingrowth or “budding” of connective tissue cells into the coating. Therefore, the ingrowth characteristics of the implant can be controlled in a targeted manner by selecting the resorbable material.

The resorbable material described above may contain additives selected in particular from the group comprising biological active substances, medical or pharmaceutical active substances, marker substances and combinations thereof. Regarding additives that may be considered, reference is made entirely to the description given hereinunder.

The coating may be formed at least as a single layer. As a rule, the coating is formed as a single layer. Basically, however, the coating can also have a two-, three- or multi-layer structure.

Especially preferably, the polyurethane coating is only formed (present) on the external surface of the prefabricated vascular prosthesis. In other words, the internal surface of the prefabricated prosthesis can be free from a polyurethane coating.

The polyurethane coating, in particular a polyurethane coating formed on the external surface of the prefabricated prosthesis, may have regions with different porosity. A polyurethane coating formed on the external surface preferably has, on its inside surface, which is opposite to the wall of the prefabricated vascular prosthesis, a lower porosity than on its outside surface. Especially preferably, the porosity of a coating formed on the external surface of the prefabricated vascular prosthesis increases from the inside surface to the outside surface of the coating, wherein the increase in porosity is preferably continuous or gradual.

Preferably, a coating formed on the external surface of the prefabricated vascular prosthesis has, on its outside surface, a three-dimensional structure, which promotes ingrowth or “budding” of connective tissue cells and/or of connective tissue and thus permits reliable attachment or anchoring of the implant in the patient's body. Preferably, on its inside surface facing the prosthesis wall, the coating has a three-dimensional structure which prevents the penetration of connective tissue cells through the wall of the prosthesis and into the lumen of the prosthesis. Narrowing of the lumen of the prosthesis can thus be avoided. Preferably, however, on its inside surface facing the prosthesis wall, the coating has a three-dimensional structure that permits penetration of low-molecular compounds through the wall of the prosthesis. In this way, for example, nutrients, biological active substances and/or medical active substances can get into the lumen of the prosthesis. Penetration of low-molecular compounds into the lumen of the prosthesis may, for example, be desirable to accelerate formation of a neointima or dispersion of a thrombus in the lumen of the prosthesis or to prevent formation of a thrombus.

Further preferably, the polyurethane coating is only formed (present) on the internal surface of the prefabricated vascular prosthesis. In this instance, the coating is preferably formed as a smooth and in particular solid layer. This can minimize the risk of blood constituents, in particular thrombocytes, fibrinogen, thrombin and the like, adhering to the inside surface of the prosthesis and possibly leading to an embolism. In other words, this structure may facilitate prevention of thrombotic occlusions.

The polyurethane coating, in particular a polyurethane coating formed on the internal surface of the prefabricated prosthesis, may be formed as a film, in particular a cast film or sprayed film, preferably a sprayed film. A film formed on the internal surface of the prefabricated prosthesis advantageously reduces the risk of thrombosis as it is significantly more difficult for blood constituents to adhere when the internal surface of the prefabricated vascular prosthesis has a lining in the form of a film. A film formed on the external surface of the prefabricated vascular prosthesis advantageously prevents undesirable penetration of body cells, in particular connective tissue cells, through the wall of the prefabricated prosthesis and into the lumen of the prosthesis.

A coating formed on the external surface of the prefabricated vascular prosthesis may be formed on its inside surface facing the prosthesis wall as a film, in particular a cast film or sprayed film, preferably a sprayed film. As a result, undesirable penetration of body cells, in particular connective tissue cells, and/or connective tissue through the prosthesis walls and into the lumen of the prosthesis can similarly be prevented. On its outside surface, i.e., the side facing the surrounding region of tissue, the coating instead preferably has a nonwoven structure, in particular a sprayed nonwoven structure, which promotes the ingrowth or “budding” of connective tissue cells and/or connective tissue.

As a rule, parts of the wall of the prefabricated prosthesis, in particular fibers, threads, yarns and the like, are connected, in particular glued, to the coating.

Preferably, the polyurethane coating, in particular a polyurethane coating formed on the external surface, comprises a nonwoven structure, preferably a sprayed nonwoven structure or is formed of such a structure. Basically, a nonwoven-like structure of the coating promotes the ingrowth or “budding” of connective tissue cells and/or connective tissue and thereby provides reliable anchoring of the implant in the patient's body.

The coating preferably has a proportion between 1 and 90 wt. % (weight percent), in particular 10 and 80 wt. %, preferably 20 and 70 wt. %, based on the total weight of the implant.

The coating preferably has a layer thickness between 0.001 and 2 mm, in particular 0.05 and 2 mm, preferably 0.1 and 1 mm, more preferably 0.2 and 0.8 mm A polyurethane coating formed on the external surface of the prefabricated vascular prosthesis preferably has a layer thickness between 0.05 and 2 mm, in particular 0.1 and 1 mm, preferably 0.2 and 0.8 mm A polyurethane coating formed on the internal surface of the prefabricated vascular prosthesis preferably has a layer thickness between 1 and 300 μm, in particular 5 and 200 μm, preferably 10 and 100 μm.

The polyurethane coating may penetrate into the prefabricated vascular prosthesis to a depth of 1 to 300 μm, in particular 5 to 200 μm, preferably 20 to 100 μm, measured from the internal and/or external surface, preferably internal surface of the prefabricated vascular prosthesis. In this way it is possible to form a solid composite structure between the polyurethane coating and the prefabricated vascular prosthesis. In particular this can prevent undesirable or premature detachment of the polyurethane coating from the prefabricated vascular prosthesis.

The coating can have a regular and/or irregular fibrous structure, in particular with respect to fiber diameters and/or fiber lengths. The coating preferably has fibers with a diameter from 0.01 to 20 μm, in particular 0.01 to 10 μm, preferably 0.1 to 10 ∥m, in particular 0.1 to 5 μm, more preferably 0.5 to 5 μm, in particular 0.5 to 3 μm.

The polyurethane coating may be formed on the internal and external surfaces of the prefabricated vascular prosthesis. In this instance, it is especially advantageous if the coating on the internal surface of the prefabricated prosthesis is formed at least partially, preferably completely, as film, in particular cast film or sprayed film, preferably sprayed film, and the coating on the external surface of the prefabricated prosthesis is formed at least partially, preferably completely, as nonwoven structure, preferably sprayed nonwoven structure. With respect to further features and advantages, reference is made entirely to the preceding description.

The polyurethane is preferably a thermoplastic polyurethane. Particularly advantageously, the polyurethane is an aliphatic and in particular linear polyurethane. Preferably, the polyurethane is a polyurethane that is soluble in organic solvents. Furthermore, the polyurethane can be a noncrosslinked polyurethane. The polyurethane can be formed from macromolecular diols and/or low-molecular diols and suitable diisocyanates. Basically, aromatic or aliphatic diols and aromatic or aliphatic diisocyanates can be used for production of the polyurethane. Preferably, the polyurethane is formed from aliphatic diols and aliphatic diisocyanates. Especially preferred macro-molecular diols are based on a polycarbonate main structure. An example of such a diol is 1,6-hexanediolpolycarbonate. Suitable low-molecular diols can be selected from the group comprising 2,2,4-trimethylhexanediol, 2,4,4-trimethylhexanediol, 1,4-butanediol and combinations thereof. Preferred aliphatic diisocyanates are hexamethylene diisocyanate, cyclohexyl diisocyanate and/or dicyclohexylmethyl diisocyanate.

The polyurethane may be a polyurethane copolymer.

The polyurethane may be selected from the group comprising aliphatic polycarbonate polyurethanes, aromatic polycarbonate polyurethanes, polyester polyurethanes, polysiloxane polyurethanes, silicone-polycarbonate polyurethanes, polyether polyurethanes, silicone-polyether polyurethanes, copolymers thereof and combinations, in particular blends, thereof. Furthermore, the polyurethane can have a molecular weight from 5000 to 100 000 dalton, preferably 20 000 to 40 000 dalton.

Preferably, the prefabricated vascular prosthesis is a textile prosthesis. The wall of the prefabricated vascular prosthesis is preferably free from a nonwoven structure, in particular free from a sprayed nonwoven structure. Especially preferably, the prefabricated vascular prosthesis is a woven or knitted prosthesis.

Furthermore, it is preferable for the prefabricated vascular prosthesis to comprise a different material than polyurethane. In particular, the prefabricated vascular prosthesis is formed from a different material than polyurethane. Preferably, the prefabricated prosthesis is formed from a nonresorbable material, as a rule a nonresorbable polymer, in particular copolymer. Suitable materials for the prefabricated vascular prosthesis can be selected from the group comprising polyesters, polyamides, polyethylene, polypropylene, polyvinylidene difluoride, polychlorotrifluoroethylene, polyhexafluoropropylene, polytetrafluoropropylene, perfluoroalkoxyvinylether, polytetrafluoroethylene, in particular expanded polytetrafluoroethylene (ePTFE), copolymers thereof and combinations, in particular blends, thereof. Preferred polyesters are polyethylene terephthalate (PET) and/or polybutylene terephthalate (PBT). Polyethylene terephthalate (PET) is especially preferred owing to its good biocompatibility and its sufficient long-term stability. Examples of suitable copolymers can be selected from the group comprising vinylidene difluoride-hexafluoropropylene copolymer, vinylidene difluoride-tetrafluoroethylene copolymer, hexafluoropropylene-tetrafluoroethylene copolymer, vinylidene difluoride-hexafluoropropylene-tetrafluoroethylene copolymer and combinations, in particular blends, thereof.

It is especially preferable if the prefabricated vascular prosthesis is formed or produced from polytetrafluoroethylene, in particular expanded polytetrafluoroethylene (ePTFE).

Preferably, the polyurethane coating is formed only on the internal surface of a prefabricated vascular prosthesis made from polytetrafluoroethylene, in particular expanded polytetrafluoroethylene (ePTFE). Advantageously, the polyurethane coating partially penetrates into the node and fibril structure of a prefabricated vascular prosthesis made from ePTFE.

The implant preferably has a porosity from 0 to 1000 ml air/min/cm², in particular 1 to 500 ml air/min/cm², at a pressure difference of approx. 1.2 kPas.

Furthermore, it is preferable if the implant has a radial tear strength between 1 and 100 N/mm, in particular 5 and 50 N/mm, preferably 10 and 30 N/mm.

Preferably, the implant is formed essentially completely from nonresorbable materials. A possible exception to this is optionally additives, in particular active substances, which can be contained in the implant, and will be considered in more detail later. The advantage of an implant that is formed exclusively from nonresorbable materials is that it causes milder tissue reactions after implantation. Thus, the breakdown or degradation of resorbable materials is as a rule accompanied by inflammatory processes which can lead to a slowing of tissue integration of implants, especially vascular prostheses. This can be a disadvantage mainly for the initial healing after implantation.

The implant may essentially preferably be completely free from materials of xenogenous, in particular bovine, equine and/or porcine, origin. A possible exception is optionally additives which can be contained in the implant and are considered in more detail later. In this way undesirable introduction of animal disease pathogens into the patient's body can be avoided.

The implant may be pleated as a rule in the form of pleated folds. The pleating can, for example, be wave-shaped, preferably as encircling transverse folds or can run spirally or helically along the external surface of the implant.

The implant may have a wall thickness (including the polyurethane coating) between 0.05 and 3 mm, in particular 0.1 and 2.0 mm, preferably 0.5 and 1.5 mm Moreover, the implant, in particular the prefabricated vascular prosthesis, can have an inside diameter between 1 and 50 mm, in particular 4 and 40 mm, preferably 6 and 38 mm.

To increase buckling stability of the implant, the implant may have reinforcements, which preferably run along the external surface of the implant. Preferably, the implant has spiral or helical reinforcements on its external surface, in particular in the form of a wire or thread, for example, a polypropylene thread.

As already mentioned, the implant, in particular the prefabricated vascular prosthesis, the coating and/or resorbable materials optionally present in pores of the coating, can have additives, in particular markers and/or active substances, preferably biological and/or medical or pharmaceutical active substances. Suitable additives are preferably selected from the group comprising cellular growth factors, cellular differentiation factors, cellular adhesion factors, cellular recruiting factors, antimicrobial, in particular antibiotic, substances, disinfectants, antiinflammatory substances, antithrombogenic substances or blood coagulation inhibitors, carrier substances, bone components, X-ray contrast agents and combinations thereof.

As antimicrobial substances, consideration is preferably given to antimicrobially effective metals, metal alloys and/or metal salts, in particular metal oxides. Suitable antimicrobial substances can, for example, be selected from the group comprising copper, zinc, tantalum, titanium, cobalt, iron, palladium, platinum, iridium, silver, gold, salts, in particular oxides, thereof and combinations, in particular alloys, thereof.

Preferred antithrombogenic substances are selected from the group comprising antithrombin III, hirudin, heparin, heparan sulfate, certoparin, dalteparin, enoxaparin, nadroparin, reviparin, tinzaparin, dabigatran, fondaparinux, lepirudin, rivaroxaban, calcium complexing agents, for example, citrate and/or EDTA and combinations thereof.

A preferred X-ray contrast agent is barium sulfate.

A suitable bone component is, for example, calcium phosphate.

The implant may thus be intended for the release of active substances (drug delivery device).

The implant may be sterilized and in particular is in packaged form. Ethylene oxide is preferably used for sterilization of the implant.

We further provide a method of production of a tubular or hose-shaped implant to replace natural blood vessels, wherein a prefabricated vascular prosthesis having an internal surface, external surface and a wall is coated with polyurethane on the internal and/or external surface.

Only the internal surface of the prefabricated prosthesis may be coated with polyurethane. Alternatively, only the external surface of the prefabricated prosthesis is coated with polyurethane. For further details and advantages reference is made entirely to the previous description.

Preferably, prior to coating with polyurethane, the prefabricated vascular prosthesis is mounted on a preferably rotatable mandrel. With a mandrel of rotatable design, especially advantageously, uniform coating of the clamped vascular prosthesis with polyurethane is possible. The mandrel is as a rule rotationally symmetrical, in particular rod-shaped or cylindrical. The mandrel can, for example, be formed from a metal, steel or plastic, in particular polyethylene or polyvinyl alcohol. The mandrel can moreover have a diameter between 1 and 50 mm, in particular 4 and 40 mm, preferably 6 and 38 mm Separation of the mandrel and the coated, prefabricated vascular prosthesis can be facilitated by coating the surface of the mandrel before mounting the vascular prosthesis with a film or a hose, for example, a latex hose.

Especially preferably, before coating with polyurethane, in particular after mounting on a preferably rotatable mandrel, the prefabricated vascular prosthesis is contacted with a suitable adhesion promoter. For this, an adhesion promoter can be poured over, painted on with a brush, sprayed or impregnated in the prefabricated vascular prosthesis. Preferably, the vascular prosthesis is impregnated with the adhesion promoter. The adhesion promoter can be in the form of a liquid dispersion, solution or suspension. The adhesion promoter is typically prepared using organic solvents. Suitable solvents can, for example, be selected from the group comprising dichloromethane, chloroform, acetone, isopropanol and mixtures thereof. Use of a solution of polyurethane, as a rule an organic polyurethane solution, as adhesion promoter is preferred. The polyurethane solution preferably has a proportion of polyurethane between 1 and 10 wt. %, based on the total weight of the polyurethane solution. The use of a polyurethane solution as adhesion promoter has the advantage that this provides particularly good adhesion of the polyurethane coating on the wall of the prefabricated vascular prosthesis.

Particularly advantageously, the coating of the prefabricated vascular prosthesis with polyurethane is carried out before the previously prepared vascular prosthesis has dried completely after contacting with the adhesion promoter. If the prefabricated vascular prosthesis is coated with several layers of polyurethane, it can moreover be advantageous if the vascular prosthesis is optionally contacted several times with an adhesion promoter between the individual coating steps. The coating with polyurethane is preferably carried out by applying, in particular casting, immersing, dipping, soaking or spraying, a solution of polyurethane on the prefabricated vascular prosthesis. Coating of the prefabricated vascular prosthesis by casting can be carried out with or without additional pressure. As an alternative, the prefabricated vascular prosthesis can also be dipped in a polyurethane solution. Coating of the prefabricated vascular prosthesis is preferably carried out by spraying a polyurethane solution on the prefabricated vascular prosthesis. For this, the polyurethane solution can, for example, be sprayed by compressed air toward the prefabricated vascular prosthesis. This can be done using a suitable spraying device, for example, a spray-gun. Basically, it is also possible to use liquid dispersions or suspensions of polyurethane for coating the prefabricated vascular prosthesis. This can, for example, be envisaged when the prefabricated vascular prosthesis is to be coated with polyurethane by a dipping technique. Generally, however, polyurethane solutions are preferred.

Preferably, the prefabricated vascular prosthesis is coated with polyurethane while tumbling the vascular prosthesis.

For coating the prefabricated vascular prosthesis, 10 to 1000 application cycles, in particular 50 to 500 application cycles, preferably 80 to 300 application cycles, may be performed.

The polyurethane may be applied at different distances, in particular at continuously increasing distances, from the prefabricated vascular prosthesis.

Application, preferably spraying, of a polyurethane solution onto the prefabricated vascular prosthesis may take place from a distance that permits fiber formation of the polyurethane from the solution as it travels the application distance, preferably spraying distance. In this way, a nonwoven-like polyurethane coating can be formed on the prefabricated vascular prosthesis. Preferably, the application distance, in particular spraying distance, is varied during application, in particular spraying. Preferably, the application distance is increased continuously during application.

The resultant nonwoven-like coating produced on the prefabricated vascular prosthesis possesses a three-dimensional structure whose porosity preferably increases continuously toward the external surface of the coating. A polyurethane solution may be applied, preferably sprayed, at the beginning of coating at a distance that does not permit fiber formation of the polyurethane from the solution as it travels the application distance, preferably spraying distance. As the coating operation continues, the distance is then preferably increased continuously so that fiber formation of the polyurethane from the solution becomes possible as it travels the application distance, preferably spraying distance. In this way, a coating can be formed on the prefabricated vascular prosthesis, being formed on its internal surface facing the prosthesis wall as a sprayed film and on its external surface possessing a sprayed structure, with the porosity of the sprayed structure preferably increasing continuously toward the external surface of the coating.

Basically, the application of a polyurethane solution can be carried out at a distance from the prefabricated vascular prosthesis between 5 and 75 cm, in particular 8 and 50 cm. If the coating is to have a non-woven structure, application of the polyurethane solution can take place at a distance from the prefabricated vascular prosthesis between 10 and 75 cm, in particular 15 and 50 cm. If, however, the coating is to be formed as a film on the prefabricated vascular prosthesis, then application of the polyurethane solution preferably takes place at a distance from the previously prepared vascular prosthesis between 1 and 45 cm, in particular 4 and 42 cm. The distances stated in this paragraph also depend in particular on the viscosity of the polyurethane solution used and the molecular weight of the polyurethane used.

After coating the internal and/or external surface of the prefabricated vascular prosthesis, the implant may be dried.

For further features and advantages of the method, reference is made to the previous description of the tubular implant.

The implant can generally be used for replacing thoracic, abdominal and/or peripheral blood vessels, preferably arterial blood vessels.

Finally, we provide for the use of polyurethane for the production of a tubular implant. With respect to further features and advantages, reference is made entirely to the preceding description.

Further features and advantages can be seen from the following description of preferred examples in conjunction with the drawings. The features can in each case be realized individually or in combination with one another. All drawings are made with express reference to the contents of this description.

### EXAMPLES

1. Production of an Implant with a Polyurethane Coating on the External Surface

For the production of an implant, a prefabricated knitted prosthesis made of polyethylene terephthalate (PET) was pulled onto a polyethylene rod. Then, the mounted, prefabricated vascular prosthesis was impregnated with a polyurethane solution which contained a proportion of polyurethane of approx. 1 wt. %, based on the total weight of the polyurethane solution. Next, before it had dried completely, the prosthesis was finished by spraying with a polyurethane solution under the conditions presented below in Table 1. The prefabricated vascular prosthesis was sprayed using a polyurethane solution having a proportion of polyurethane of approximately 10 wt. %, based on the total weight of the solution.

TABLE 1
Production conditions for a vascular prosthesis

| Parameter | Setting |
|---|---|
| Product length [mm] | 750 (clamped) |
| Number of phases [number] | 4 |
| Product arbor [rpm] | 200 |
| Spraying head feed [m/min] | 2 |
| Gun | |
| Gun height [mm] | −6 |
| Gun raster spray jet [raster] | 18 |
| Nozzle size [mm] | 0.3 |
| Spraying distance [mm] | 50/420/200/420 |
| Material pressure [bar] | 0.41/0.16 |
| Gun angle [±degrees] | |
| left | 45 |
| right | 45 |
| Rotary speed of material | 280/130/130/130 |
| pump [rpm] | |
| Pressure [bar] | |
| fan jet | 0 |
| circular-section jet | 3 |
| Cycles [number] | 20/25/15/10 |
| Countershaft angle [degrees] | 0 |
| PE tube [mm] | 6.7 OD |
| Climatic conditions on rod | 98%/50° C./2 h |
| Miscellaneous | before spraying, impregnated with 1% |
| | polyurethane solution, 2 layers sprayed, |
| | impregnated again with 1% |
| | polyurethane solution |

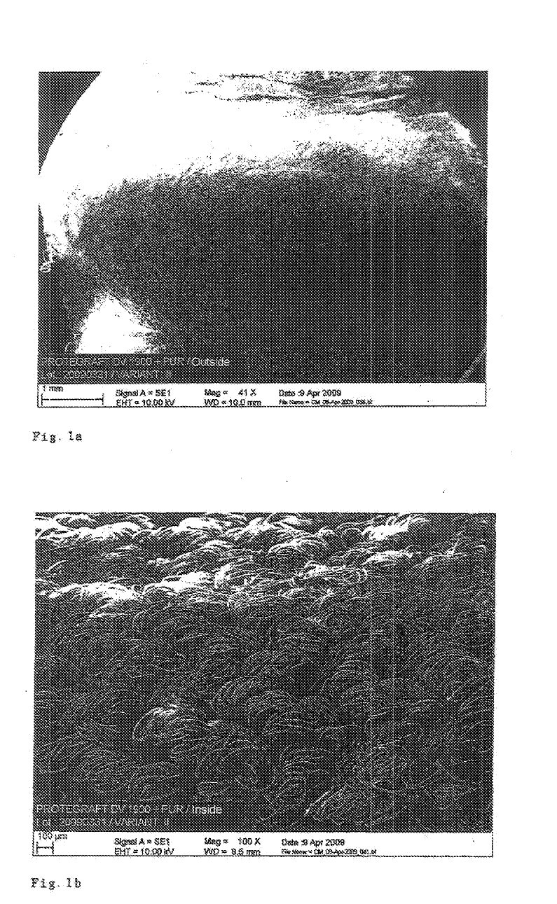

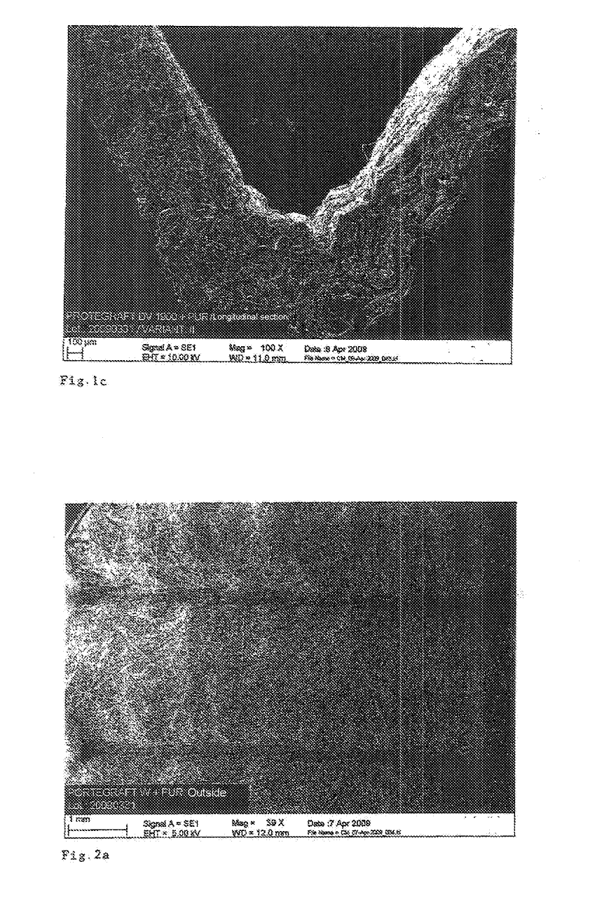

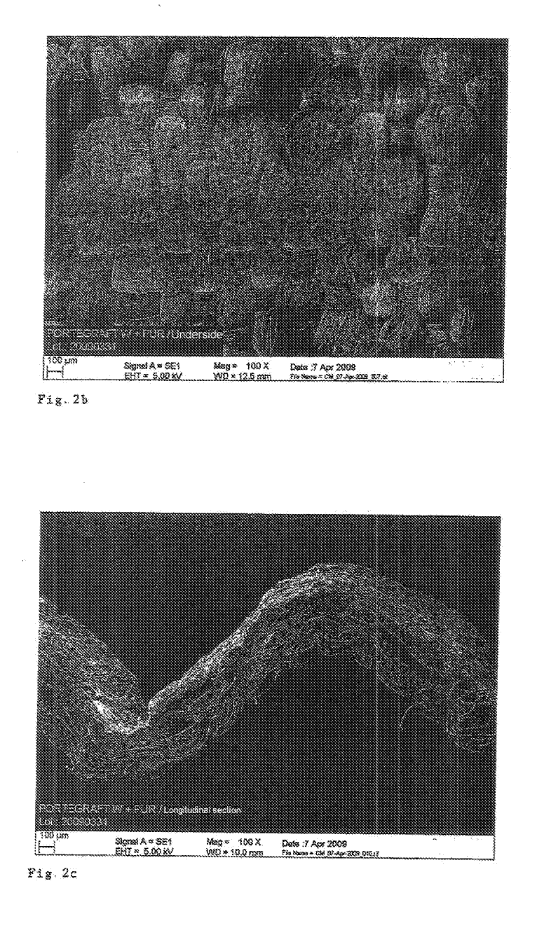

Production of an implant based on a woven vascular prosthesis was carried out correspondingly, starting from a previously prepared woven vascular prosthesis. The results are shown in the form of photographs in FIGS. 1 and 2.

2. Production of an Implant with a Polyurethane Coating on the Internal Surface

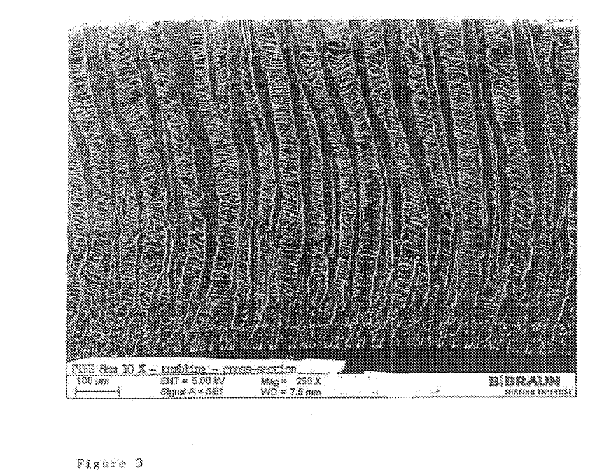

A prefabricated vascular prosthesis made of expanded polytetrafluoroethylene (ePTFE) was clamped in a holder which was equipped above and below with sealable hose outlets. Then, the clamped vascular prosthesis was filled with a polyurethane solution which contained a proportion of approx. 10 wt. %, based on the total weight of the polyurethane solution to about half the total length of the prosthesis. Then, the hose outlets were sealed above and below the vascular prosthesis. The prosthesis was tumbled for approximately 1 minute. After tumbling, the vascular prosthesis was returned to a vertical position and the lower outlet opened so that the polyurethane solution could drain away. The prosthesis was then cut away from the lower hose outlet and dried, freely suspended, for 24 hours. The resulting implant is shown in FIG. 3.

1. A tubular implant that replaces natural blood vessels comprising a prefabricated vascular prosthesis with an internal. surface an external surface and a wall, wherein the internal and/or external surface of the prefabricated vascular prosthesis has a polyurethane-coating.

2. The tubular implant as claimed in claim 1, wherein the polyurethane coating is only formed on the external surface of the prefabricated vascular prosthesis.

3. The tubular implant as claimed in claim 1, wherein the polyurethane coating is only formed on the internal surface of the prefabricated vascular prosthesis.

4. The tubular implant as claimed in claim 1, wherein the polyurethane coating comprises a sprayed nonwoven structure.

5. The tubular implant as claimed in claim 1, wherein the polyurethane coating is formed as a smooth, nondetachable, sprayed film layer.

6. The tubular implant as claimed in 1, wherein the polyurethane coating has a layer thickness between 0.05 and 2 mm.

7. The tubular implant as claimed in claim 1, wherein the polyurethane coating has a layer thickness between 1, and 300 μm.

8. The tubular implant as claimed in claim 1, wherein the polyurethane coating penetrates to a depth of 1 to 300 μm into the wall of the prefabricated vascular prosthesis when measured from the internal surface of the prefabricated vascular prosthesis.

9. The tubular implant as claimed in claim 1, wherein the implant is formed essentially, completely from nonresorbable materials.

10. The tubular implant as claimed in claim 1, wherein the implant is completely free from materials of xenogenous; origin.

11. The tubular implant as claimed in claim 1, wherein the prefabricated vascular prosthesis is a woven or knitted, textile-vascular prosthesis, and formed from a material other than polyurethane, selected from the group consisting of polyesters, polyamides, polyethylene, polypropylene, polychlorotrifluoroethylene, polyvinylidene difluoride, polyhexafluoropropylene, perfluoroalkoxyvinylether, polytetrafluoropropylene, polytetrafluoroethylene, copolymers thereof and combinations thereof.

12. The tubular implant as claim 1, wherein the polyurethane coating is only formed on the internal surface of the prefabricated vascular prosthesis and the prefabricated vascular prosthesis is produced from expanded polytetrafluoroethylene (ePTFE).

13. The tubular implant as claimed in claim 1, wherein the implant has a porosity from 0 to 1000 ml air/min/cm², at a pressure difference of 1.2 kPas.

14. The tubular implant as claimed in claim 1, wherein the implant has a radial tear strength of 1 to 100 N/mm.

15. A method of producing a tubular implant that replaces natural blood vessels as claimed in claim 1, wherein the prefabricated vascular prosthesis with the internal and external surfaces and the wall, is coated with polyurethane on the internal and/or external surface.

16. The method as claimed in claim 15, wherein the prefabricated vascular prosthesis is clamped on a rotatable mandrel prior to coating with polyurethane.

17. The method as claimed in claim 15, wherein prior to coating with polyurethane, the prefabricated vascular prosthesis is contacted with an adhesion promoter, comprising a solution of polyurethane with a polyurethane concentration of 1 to 10 wt. %, based on the total weight of the polyurethane solution.

18. The method as claimed in claim 15, wherein coating is carried out by ;casting, immersing, dipping, soaking or spraying a solution of polyurethane on the prefabricated vascular prosthesis.

19. The method as claimed in claim 15, wherein coating, is carried out while tumbling the prefabricated vascular'prosthesis.
